(19) United States
(12) Patent Application Publication
(10) Pub. No.: US 2024/0245791 A1
(43) Pub. Date: Jul. 25, 2024

(54) METHODS AND COMPOSITIONS FOR LOCALIZATION OF GROWTH FACTORS

(71) Applicant: Arizona Board of Regents on Behalf of the University of Arizona, Tucson, AZ (US)

(72) Inventors: Jason Wertheim, Tucson, AZ (US); Kyle Koss, Tucson, AZ (US); Yangxi Liu, Tucson, AZ (US)

(21) Appl. No.: 18/566,564

(22) PCT Filed: Jun. 2, 2022
(86) PCT No.: PCT/US22/32018

§ 371 Date: Dec. 1, 2023

Related U.S. Application Data
(60) Provisional application No. 63/196,109, filed on Jun. 2, 2021.

(51) Int. Cl. A61K 47/64; A61K 38/18; A61K 47/54

(52) U.S. Cl. CPC A61K 47/64; A61K 38/1866; A61K 38/1891; A61K 47/545

(57) ABSTRACT

The present invention features compositions and methods for the localization and concentration of various growth factors (e.g., vascular endothelial growth factor (VEGF), or an angiopoietin-1 (ANG1) protein, or an angiopoietin-2 (ANG2) protein) within a scaffold structure. The present invention may also be used to promote the rebuilding of vasculature on de-celled organs (e.g., a kidney) or artificial organs.

### CROSS-REFERENCES TO RELATED APPLICATIONS

This application claims benefit of U.S. Provisional Application No. 63/196,109 filed Jun. 2, 2021, the specification of which is incorporated herein in their entirety by reference.

### STATEMENT REGARDING FEDERALLY SPONSORED RESEARCH OR DEVELOPMENT

This invention was made with government support under Grant No. DK113168 awarded by National Institutes of Health. The government has certain rights in the invention.

### REFERENCE TO A SEQUENCE LISTING

Applicant asserts that the information recorded in the form of an Annex C/ST.25 text file submitted under Rule 13ter.1(a), entitled >>>UNIA_21_16_PCT_Sequence_Listing_ST25<<<, is identical to that forming part of the international application as filed. The content of the sequence listing is incorporated herein by reference in its entirety

### FIELD OF THE INVENTION

The present invention features compositions and methods for the localization and concentration of various growth factors (e.g., vascular endothelial growth factor (VEGF)) within a scaffold structure (e.g., a scaffold for creating an artificial organ).

### BACKGROUND OF THE INVENTION

Angiogenesis is the development of new blood vessels. It is the initial and one of the hardest stages when creating a scaffold to create an artificial organ. Without correct vasculature and/or blood circulation, an artificial organ cannot survive. One critically important factor in angiogenesis is vascular endothelial growth factor (VEGF). VEGF is a signaling protein that promotes the growth of new blood vessels. However, with minimal binding sites on 3D scaffold structures, there is usually not enough VEGF present to properly support cells within the scaffold for long term survival.

Therefore, the present invention has developed compositions and methods to increase the concentration of VEGF (and other growth factors) within 3D scaffolds as well as allow for proper localization of VEGF (and other growth factors) on the scaffold.

### BRIEF SUMMARY OF THE INVENTION

It is an objective of the present invention to provide compositions and methods that allow for the localization and concentration of growth factors (e.g., vascular endothelial growth factor (VEGF)) to support long term survival of cells within a culture (e.g., 2D or 3D cultures), as specified in the independent claims. Embodiments of the invention are given in the dependent claims. Embodiments of the present invention can be freely combined with each other if they are not mutually exclusive.

In some embodiments, the present invention features a composition comprising a growth factor (GF) protein conjugated to one or more targeting moieties. In some embodiments, the growth factor (GF) is a vascular endothelial growth factor (VEGF) protein, or an angiopoietin-1 (ANG1) protein, or an angiopoietin-2 (ANG2) protein. In some embodiments, the targeting moiety is a collagen binding protein (CBP) or a hyaluronic acid binding peptide (HBP).

In some embodiments, the present invention features a composition comprising a growth factor (GF) conjugated to one or more avidin or streptavidin proteins. In other embodiments, the present invention features a composition comprising a vascular endothelial growth factor (VEGF) protein conjugated to avidin or streptavidin proteins. The compositions described herein may further comprise a targeting moiety (e.g., collagen binding protein (CPB)) conjugated to biotin. In other embodiments, the composition further comprises multi-antigen peptide (MAP) comprising four lysines conjugated to four targeting moiety (e.g., collagen binding protein (CPB).

The present invention may further feature a method of forming a vascular system in an artificial organ (e.g., an artificial kidney). The method may comprise introducing a composition as described herein to the artificial organ (e.g., the artificial kidney). The present invention may also feature a method of localizing a growth factor (e.g., vascular endothelial growth factor (VEGF) protein) to a scaffold. The method comprises introducing a composition as described herein to a scaffold.

One of the unique and inventive technical features of the present invention is the use of a targeting moiety (e.g., collagen binding protein (CBP)) conjugated to a growth factor (e.g., vascular endothelial growth factor (VEGF)). Without wishing to limit the invention to any theory or mechanism, it is believed that the technical feature of the present invention advantageously provides for multiple targeting moieties (e.g., CBP) to be conjugated to a GF (e.g., VEGF) which allows higher concentrations of the GF at specific locations (e.g., localized at sites with collagen IV).

Furthermore, the prior references teach away from the present invention. For example, VEGF has previously been linked to CBP through recombinant expression. However, recombinant expression only allows for one CBP to be linked to VEGF because recombinant technologies only allow proteins to be designed in a linear order (i.e., synthesized from an N- to C-terminus). Therefore, recombinant expression does not allow for multiple targeting moieties (e.g., CBP) to be conjugated to a GF (e.g., VEGF) or for branching of the multiple targeting moieties from a GF. Without wishing to limit the present invention to any theory or mechanism it is believed that branching may result in superior protein-protein interactions. Furthermore, the targeting moieties are conjugated in the areas where folding allows for external interactions such as binding. This is largely because the chemistry conditions (pH and saline salts) are maintained throughout the click and NHS chemistries.

Another unique and inventive technical feature of the present invention is the use of a growth factor (e.g., vascular endothelial growth factor (VEGF)) conjugated to an avidin composition in combination with a biotin-targeting moiety-MAP (e.g., biotin-CBP-MAP) composition. Without wishing to limit the invention to any theory or mechanism, it is believed that the technical feature of the present invention advantageously provides for the ability to create self-assemblies of VEGF on collagen IV (i.e., 3D structures). None of the presently known prior references or work has the unique inventive technical feature of the present invention. Furthermore, the prior references teach away from the present invention. For example, normally only 5% of VEGF added to a scaffold will bind because it is limited to the number of collagen IV sites available. However, the present invention circumvents these limitations and allows for all of the VEGF present to bind together creating a 3D structure. The 3D structure is generated by the four biotin on the biotin-targeting moiety-MAP (e.g., biotin-CBP-MAP4) composition (i.e., a tetramer of N-terminus capped biotin-CBP) and the four avidin binding sites (avidin is a natural tetramer) on the avidin-GF (e.g., avidin-VEGF or avidin-ANG1, or avidin-ANG2). The four binding sites on each molecule (i.e., avidin and biotin) promotes intense intermolecular polymerization that is rich in both collagen IV binding sites and growth factors (e.g., VEGF, ANG1, ANG2, or heparin). As long as there is an equimolar amount of biotin and avidin, all the GF (e.g., VEGF) in the system should adhere to the collagen IV surface. This might make a lower affinity (but highly specific) peptide a total binder.

Any feature or combination of features described herein are included within the scope of the present invention provided that the features included in any such combination are not mutually inconsistent as will be apparent from the context, this specification, and the knowledge of one of ordinary skills in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1 shows non-limited examples of growth factors conjugated to targeting moieties. For example, FIG. 1 shows how a vascular endothelial growth factor (VEGF) protein is conjugated to collagen binding protein (CPB).

FIGS. 2A and 2B show an overview of the click chemistry and how established it is in bio-conjugate synthesis. The azido and alkyne (“lock and key”) groups are noted here in NHS forms, which is a group that can spontaneously conjugate to amine sites on proteins in most biological buffers.

FIG. 3 shows the two-step chemistry needed to conjugate VEGF to CPB. Step 1 comprises adding an azido to the amines on the VEGF121 protein, which there are up to 10 on lysines and the N-terminus. The NHS group is a spontaneous reactant that targets amine groups. Step two comprises using click chemistry to conjugate the azido group to the alkyne (or propargyl) group on the CBP. The CBP can be made by a company with an alkyne group on its N-terminus by solid phase synthesis.

FIG. 4 shows the optimization of adding an azido group to a protein of interest (i.e., VEGF). FIG. 4 shows the mass spectrometry (LC-TOF) of the chemistry for the conjugations of VEGF with an azido group (see FIG. 3 (1)). This is a series of optimization using bovine serum albumin (BSA), which is an inexpensive protein for the purpose of optimization. The x-axis represents the mass/charge or molecular weight of the protein, therefore shifts to the right indicate the addition of conjugate groups. The leftmost set of figures shows the addition of azido groups by molar content of azido-NHS and the rightmost set of figures shows a time-based kinetics. As azido-NHS molarity increases, the peak shifts right 200-300 m/z, suggesting 2-3 azido modifications. At 100× molar ratio this is the best, with the narrowest distribution, suggesting all the amines are modified with azido. There is no difference between 0 minutes to hours, suggesting this reaction is instantaneous.

FIG. 5 shows the optimization of the “click-chemistry” to conjugate an azido group to an alkyne group. FIG. 5 shows the mass spectrometry (LC-TOF) of the click-chemistry (see FIG. 3 (2). This is the click portion of the chemistry with BSA as a representative. The left-most shift (˜1000 m/z) in the m/z in the bottom two figures shows that CBP was added to the molecule. The peaks are also spaced apart by the same shift, suggesting a distribution of 1-4 added CBP groups. The second to last and last figure are kinetics done at 1 hour and 24 hours, and the kinetics were not different, suggesting that the reaction only needs to last 1 hour.

FIG. 6 shows the modification of VEGF in the mass spectrometry (LC-TOF) of the chemistry or the conjugations of VEGF with an azido group (see FIG. 3 (1)). This is the initial chemistry using VEGF. The reaction was performed at a 100:1 azido-NHS:VEGF molar ration overnight. The top figures are the original VEGF, the right being a close up of one peak, and the bottom figures are the azido modified VEGF. The shift is comparable to the BSA shift being 2-4 azido groups.

FIG. 7 shows a binding assay to collagen IV of the CBP-VEGF vs the CBPi-VEGF, which is a conjugate with scrambled peptide control that has the same amino acid composition. The result is measured using an ELISA for VEGF121. The binding is significantly greater than the scrambled control at all concentrations. Herein, the CBP-VEGF is a superior binder to collagen IV and is capable of being tagged by capture and detection antibodies in the assay, suggesting that this molecule still maintains structural integrity after modification.

FIG. 8 shows a binding assay to collagen IV of biotin-CBP-QK vs biotin-CBPi-QK, which is a conjugate with scrambled peptide control that has the same amino acid composition. The result is measured using Alexafluor 488 streptavidin. The binding is significantly greater than the scrambled control at all concentrations. Herein, the biotin-CBP-QK is a superior binder to collagen IV at lower concentration, which may be due to better solubility at that concentration.

FIG. 9 shows the chemistry of how a vascular endothelial growth factor (VEGF) protein is conjugated to an avidin peptide. This is a two step chemistry. (1) An azido is added to the amines on the VEGF121 protein, there are up to 10 on lysines and the N-terminus. Similarly, alkyne (or propargyl) is added to the amines of the avidin. (2) The click chemistry conjugates the azido group to the alkyne (or propargyl) group on the Avidin.

FIG. 10 shows a multi-antigen peptide (MAP). The multi-antigen peptide (MAP), can be provided by a company. However, the sequence of the biofunctional sequences CBP and the biotin group must first be specified. There are two benefits to using MAP: 1) it allows for a mixing and matching of binding peptides (e.g., CBP) with different growth factors (e.g., VEGF121) for a highly tailored engineering of a scaffolds, and 2) the tetramer of CBP can has more ability to bind to collagen IV, the tetramer of biotin can bind to avidin, and the tetramer on the VEGF-Avidin can bind to the biotin and form an intermolecular bound polymer that self assembles into a 3D layer, going beyond the 2D limitations of collagen IV binding sites.

FIG. 11 shows a binding assay to collagen IV of the CBP-MAP4 vs the CBPi-MAP4, both with VEGF-Avidin. CBPi-MAP4 has a scrambled version like the other conjugates. The MAPs were added first (overnight) and the VEGF-Avidin was added after in an equimolar amount. Only at the higher concentration, does this CBP group have better binding. This requires some optimization, and likely not all of the MAP solution should remain before the addition of the VEGF-Avidin.

FIG. 12 shows an avidin tetramer complexing with a biotin attached to a targeting moiety.

FIG. 13 shows Angiopoieitin 1-bioconjugate activity measured based on phosphorylation of Tie2 receptor

FIG. 14 shows Angiopoieitin 1-bioconjugate binding measured based on ANG1 ELISA binding to collagen IV coating for 24 hours. The last two groups are an equimolar co-treated ANG1-Avidin (ANG1A) and CBPmap4 (CBPm4).

FIG. 15 shows Heparin-bioconjugate binding measured based on biotin-AF488 streptavidin signal to collagen IV coating for 24 hours. The first four groups refer to single conjugates of peptide and heparin. B groups refer to both Heparin-Avidin (HepA) and CBPmap4 (CBPm4) being treated equimolar at the same time. The other HepA/CBPm4 group had a prior CBPm4 treatment for 24 hours prior to the addition of HepA.

### DETAILED DESCRIPTION OF THE INVENTION

Before the present compounds, compositions, and/or methods are disclosed and described, it is to be understood that this invention is not limited to specific synthetic methods or to specific compositions, as such may, of course, vary. It is also to be understood that the terminology used herein is for the purpose of describing particular embodiments only and is not intended to be limiting.

As used herein, the term “Click-Chemistry” refers to a chemical synthesis, “click” chemistry is a class of biocompatible small molecule reactions commonly used in bioconjugation, allowing the joining of substrates of choice with specific biomolecules. Click chemistry is not a single specific reaction, but describes a way of generating products that follow examples in nature, which also generates substances by joining small modular units. In many applications, click reactions join a biomolecule and a reporter molecule. Click chemistry is not limited to biological conditions: the concept of a “click” reaction has been used in pharmacological and various biomimetic applications. However, they have been made notably useful in the detection, localization and qualification of biomolecules. Furthermore, click chemistry allows for branching from a lysine/arginine and/or n-termini; numerous sequences may be used as long as the sequence has more than one lysine/arginine at an N-terminus.

As used herein, “biotin” is a small molecule with robust chemistry. Additionally, biotin can be synthesized onto amines (lysines) via solid phase chemistry. As used herein, “avidin” or “streptavidin” are large proteins (68 kDa) that strongly bind to biotin. Additionally, avidin and streptavidin are tetramers. The present invention utilizes click chemistry to conjugate avidin to a growth factor (e.g., VEGF) which allows avidin to exist as a tetramer in the compositions described herein.

As used herein, “vascular endothelial growth factor (VEGF)” is a growth factor that promotes angiogenesis and is a necessary factor for survival and differentiation of vasculature in a culture.

As used herein, “angiopoietin-1 (ANG1)” is a growth factor that promotes angiogenesis and confluent vascular walls noted in mature tissues. As used herein, “angiopoietin-2 (ANG2)” is a growth factor that has a regulatory role in angiogenesis and confluent vascular walls and functions in preventing vascular disease.

As used herein, “heparin” is a glycosaminoglycan that may be used to reduce clotting, thrombosis, and neointimal hyperplasia (i.e., scarring and constriction of vasculature in transplants).

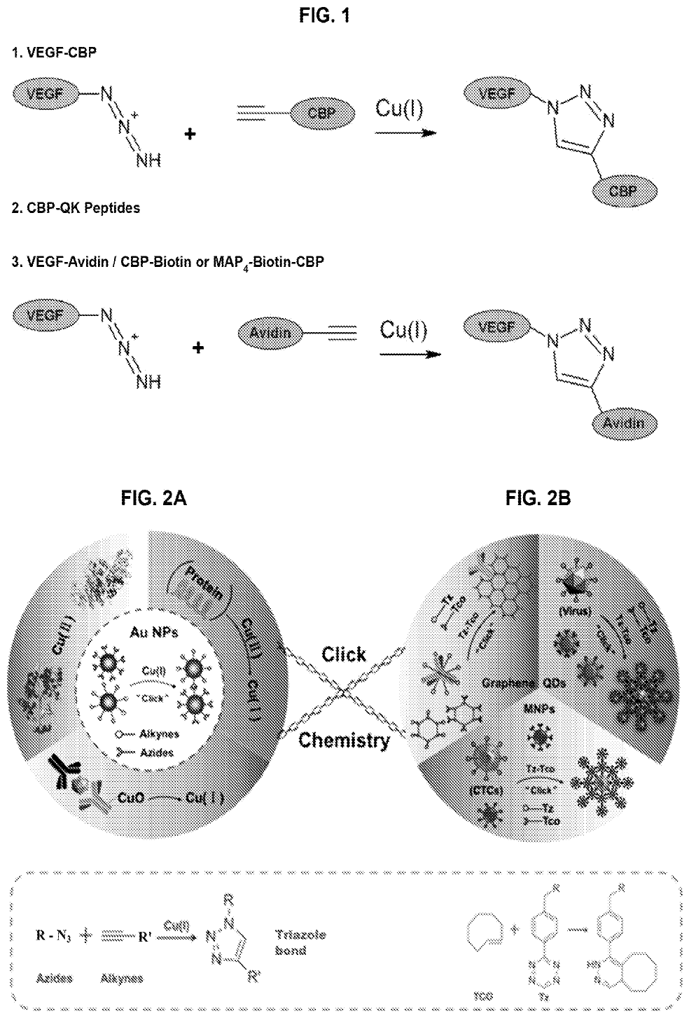

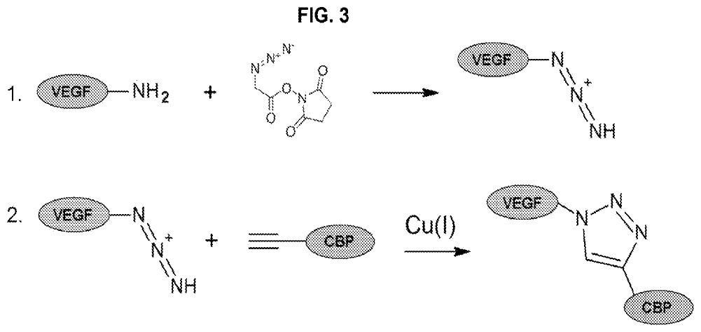

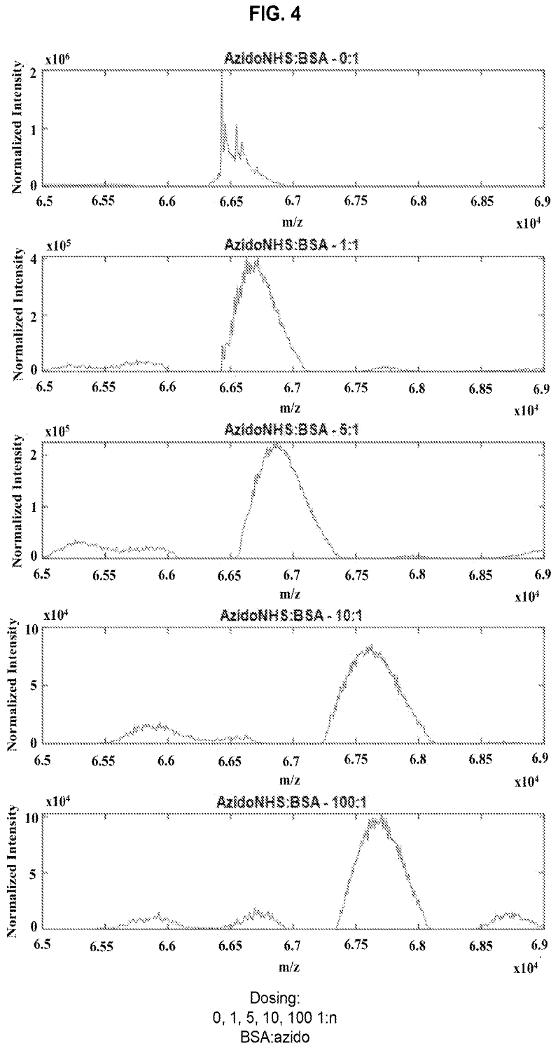

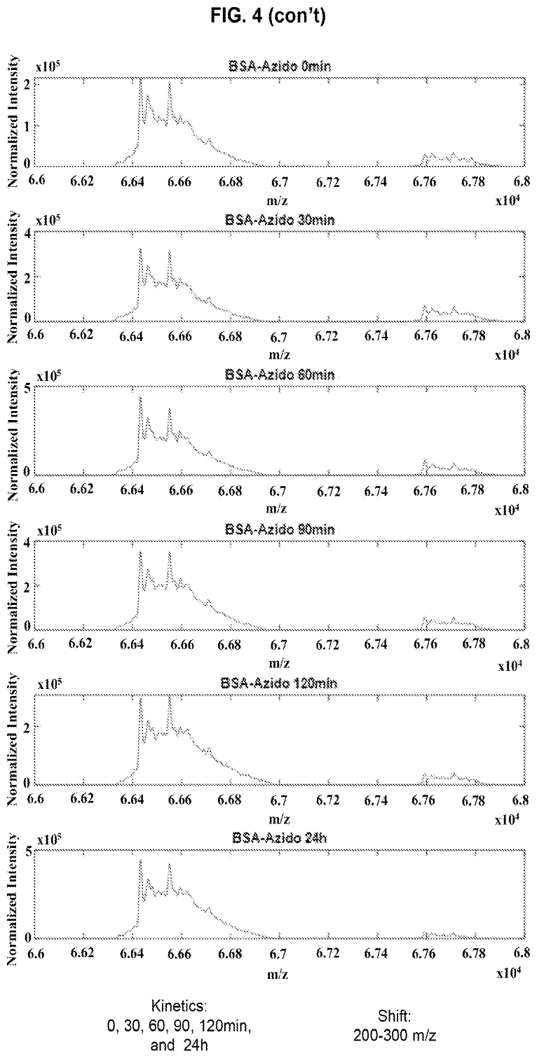

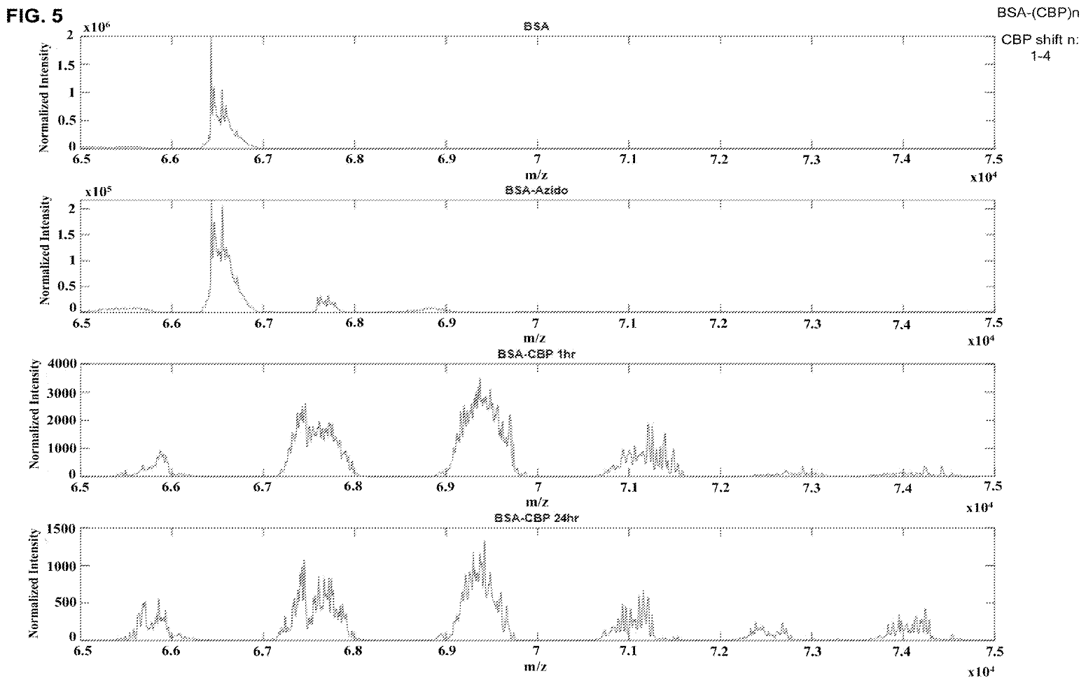

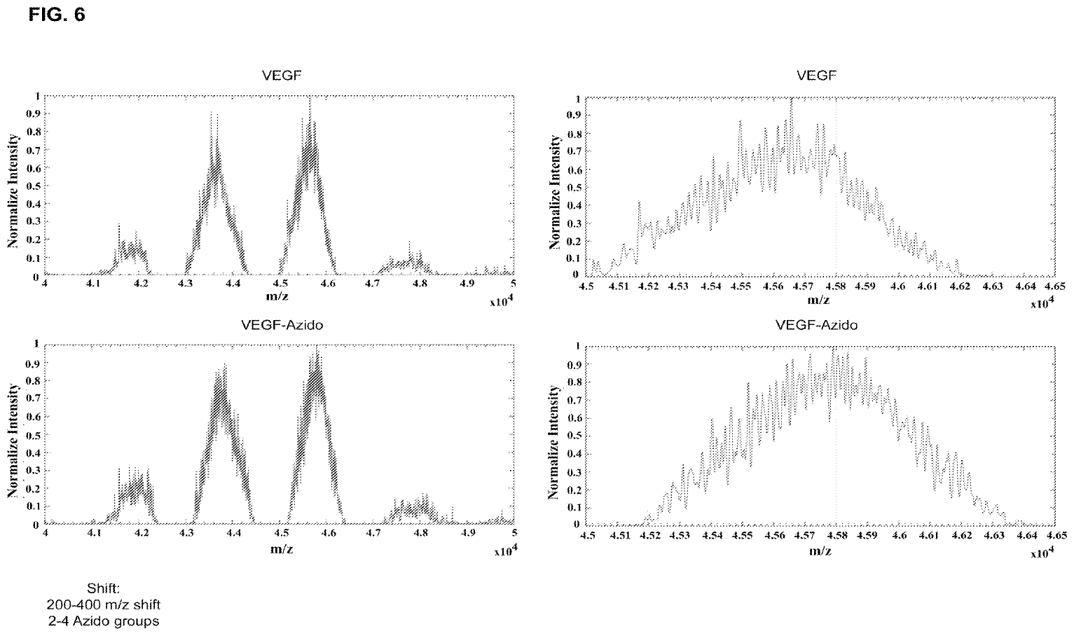

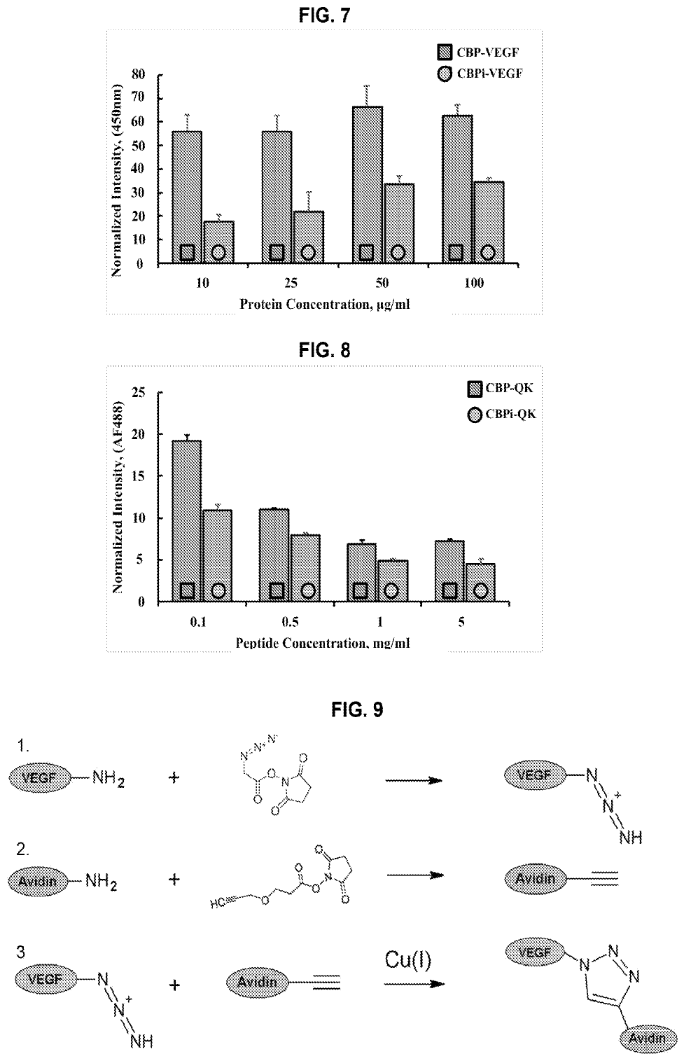

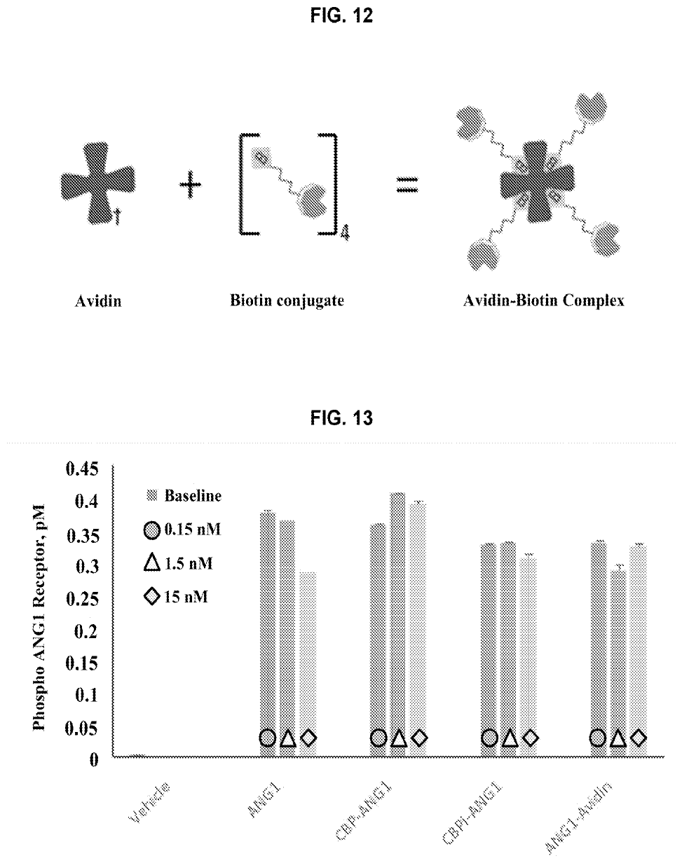

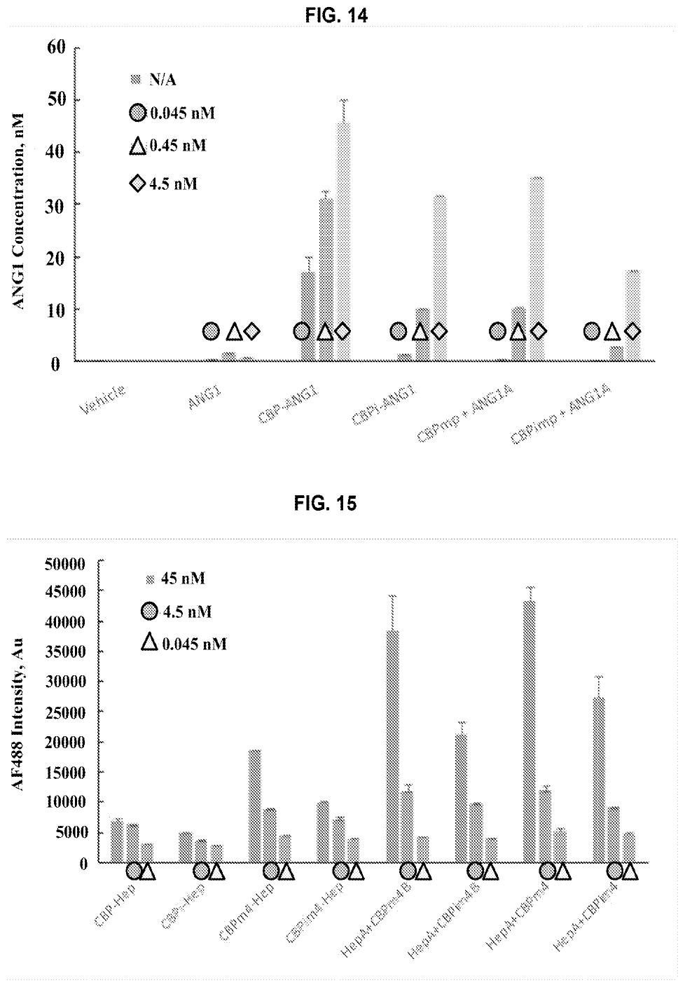

Referring now to FIGS. 1-15, the present invention features compositions and methods for the localization and concentration of various growth factors (i.e. vascular endothelial growth factor (VEGF)) within a scaffold structure.

The present invention features a composition comprising a growth factor (GF) protein conjugated to one or more targeting moieties. In some embodiments, the composition comprises a growth factor (GF) protein conjugated to two or more targeting moieties. In other embodiments, the composition comprises a growth factor (GF) protein conjugated to three or more targeting moieties. In further embodiments, the composition comprises a growth factor (GF) protein conjugated to four or more targeting moieties. The compositions described herein may comprise one targeting moiety, or two targeting moieties, or three targeting moieties, or four targeting moieties, or five targeting moieties, or six targeting moieties. Without wishing to limit the present invention to any theories or mechanisms it is believed that the number of targeting moieties that may be conjugated to a GF depends on the number of available lysine residues in the sequence, as well as the protein folding during chemistry. In some embodiments, the GF is a vascular endothelial growth factor (VEGF) protein. In some embodiments, the targeting moiety is a collagen binding protein (CBP) or a hyaluronic acid binding peptide (HBP).

In some embodiments, the composition comprises a growth factor (GF) protein conjugated to one or more CBPs, or to two or more CBPs, or to three or more CBP, or to four or more CBPs. In other embodiments, the composition comprises a growth factor (GF) protein conjugated to one or more HBPs, or to two or more HBPs, or to three or more HBP, or to four or more HBPs.

In some embodiments, the growth factor (GF) protein is conjugated to one targeting moieties (e.g., CBP or HBP). In other embodiments, the GF protein is conjugated to two targeting moieties (e.g., CBP or HBP). In some embodiments, the GF protein is conjugated to three targeting moieties (e.g., CBP or HBP). In other embodiments, the GF protein conjugated to four targeting moieties (e.g., CBP or HBP). In further embodiments, the GF protein is conjugated to more than four targeting moieties (e.g., CBP or HBP). As used herein, a “targeting moieties” refers to a protein and/or peptide that targets the composition to a specific location.

In some embodiments, the present invention features a composition comprising a VEGF protein conjugated to one CBP, or two CBPs, or three CBPs, or four CBPs.

The present invention may feature a composition comprising a VEGF protein conjugated to one or more CPB. In some embodiments, the composition comprises a VEGF protein conjugated to two or CBPs. In other embodiments, the composition comprises a VEGF protein conjugated to three or more CBPs. In further embodiments, the composition comprising a VEGF protein conjugated to four or more CBPs. In some embodiments, the present invention features a composition comprising a VEGF protein conjugated to one CBP, or two CBPs, or three CBPs, or four CBPs. In other embodiments, the present invention features a composition comprising a VEGF protein conjugated to one HBP, or two HBPs, or three HBPs, or four HBPs.

Without wishing to be bound to a particular theory or mechanism, the VEGF-CBP composition is a molecule with the capability of binding to collagen IV (peptide sequence NH2-CQDSETRTFY-COOH (SEQ ID NO: 1)) and promoting angiogenesis. The novelty of this molecule (i.e., composition) is that it is capable of targeting specific collagen IV components of a bioreactor scaffold, or injured tissues in vivo, and promoting revascularization of those areas. Compared to its alternative method of manufacturing, using expression technology, using click-chemistry the present invention has the ability to attach multiple CBP sequences (i.e., targeting moieties) per VEGF (i.e., growth factors) present on sites of the protein folded outwards, making it a more robust binding agent.

The present invention may feature a composition comprising an ANG1 or ANG2 protein conjugated to one or more CPB. In some embodiments, the composition comprises an ANG1 or ANG2 protein conjugated to two or CBPs. In other embodiments, the composition comprises an ANG1 or ANG2 protein conjugated to three or more CBPs. In further embodiments, the composition comprising an ANG1 or ANG2 protein conjugated to four or more CBPs. In some embodiments, the present invention features a composition comprising an ANG1 or ANG2 protein conjugated to one CBP, or two CBPs, or three CBPs, or four CBPs. In other embodiments, the present invention features a composition comprising an ANG1 or ANG2 protein conjugated to one HBP, or two HBPs, or three HBPs, or four HBPs.

In some embodiments, the growth factor is a VEGF protein. In some embodiments, the targeting moiety is conjugated to a primary amine of the VEGF protein. In some embodiments, the VEGF protein is an isoform of VEGF. In some embodiments, the isoform of VEGF is VEGF 121 or VEGF 165. In other embodiments, the growth factor is angiopoietin 1 (ANG1) or angiopoietin 2 (ANG2). In further embodiments, the growth factor is an angiogenic growth factor.

Without wishing to limit the present invention to any theories or mechanisms it is believed that the 3D structures of the growth factors described herein are important to their ability to bind their corresponding receptor and the function of the compositions described herein. A peptide that emulates the binding domain of a GF may be used in accordance with compositions described herein. For example, the QK peptide designed to emulate the binding site to the VEGF receptor may be used in compositions described herein.

The present invention may further feature a composition comprising a QK peptide conjugated to one or more targeting moieties. In some embodiments, the composition comprises a QK peptide conjugated to two or more targeting moieties. In other embodiments, the composition comprises a QK peptide conjugated to three or more targeting moieties. In further embodiments, the composition comprising a QK peptide conjugated to four or more targeting moieties. The present invention may also feature a composition comprising a QK peptide conjugated to one or more collagen binding protein (CPB). In some embodiments, the present invention features a composition comprising a QK peptide conjugated to two or more CPBs, or three or more CPBs, or four or more CPBs. In some embodiments, the present invention features a composition comprising a QK peptide conjugated to one or more HBPS, or two or more HPBs, or three or more HPBs, or four or more HPBs.

In some embodiments, the present invention features a portion of vascular endothelial growth factor (VEGF) protein conjugated to one or more targeting moieties. In other embodiments, the present invention features a portion of vascular endothelial growth factor (VEGF) protein conjugated to one or more collagen binding protein (CPB).

The QK peptide (NH2-KLTWQELYQLKYKGI-COOH (SEQ ID NO: 15)) is a pro-angiogenic peptide. Additionally, the QK peptide is an angiogenic peptide analogue which behaves comparably to VEGF and is designed to emulate the VEGF binding domain. When conjugated with the CBP peptide, with the sequence being biotin-CQDSETRT-KLTWQELYQLKYKGI-COOH (SEQ ID NO: 16) and the N-terminus is capped by biotin for imaging applications. The QK-CBP composition should function similarly to the VEGF-CBP composition. However, the benefit of the QK-CBP composition over the VEGF-CBP composition is that it can be made on a larger industrial scale with solid phase synthesis with higher purity.

In some embodiments, the growth factors proteins described herein are conjugated to the targeting moieties by solid phase chemistry (e.g., click chemistry). In some embodiments, the growth factors proteins described herein are conjugated to the CPBs by solid phase chemistry (e.g., click chemistry). In some embodiments, the QK peptide is conjugated to the targeting moieties by solid phase chemistry (e.g., click chemistry). In some embodiments, the QK peptide is conjugated to the CBPs by solid phase chemistry (e.g., click chemistry).

In some embodiments, the targeting moiety is a collagen binding protein (CBP; SEQ ID NO: 1). The CPB binds specifically to collagen IV, which may be present at a site of injury or at a site where cells have been stripped away. In other embodiments, the targeting moiety is human decorin collagen binding peptide (CBP; SEQ ID NO: 2). In further embodiment, the targeting moiety is bovine CBP; SEQ ID NO: 3). The bovine CPB may bind to collagen I, collagen III, collagen VI, or a combination thereof.

TABLE 1
Shows non-limiting examples of collagen bind proteins
(CPBs; i.e., targeting moieties) that may be
used in compositions described herein:

| SEQ | | Amino Acid Sequence |
|---|---|---|
| ID NO: | Peptide | (N- to C- terminal) |
| 1 | collagen binding protein (CBP) | CQDSETRTFY |
| 2 | human decorin collagen binding | RELHLNNNL |
| | peptide (CBP) | |
| 3 | bovine CBP | LRELHLNNNC |

The “N-terminal” of the peptides may further comprise a amine group (NH2) and the “C-terminal” of the peptides may further comprise a carboxyl group (COOH)

In other embodiments, the targeting moiety is a hyaluronic acid (HA) binding peptide (HBP) including but not limited to a mPep35 peptide, a 17x-3 peptide, a 6f-2 peptide, a 8h-2 peptide, a 2^{nd} de novo peptide, a 10K peptide, a 4 peptide, a 6b peptide, a 7c peptide, a BHP3 peptide, a BHP4 peptide, or a combination thereof (see Table 2 for sequences).

Table 2: Shows non-limiting examples of hyaluronic acid (HA) binding peptides (i.e., targeting moieties) that may be used in compositions described herein. The HA binding domains (shown in column 4) are critical to the binding to hyaluronic acid (HA). These peptides bind HA based secondary structural arrangement rather than a linear primary sequence.

| SEQ ID | | | HA binding domains [B(X₇)B]: |
|---|---|---|---|
| NO: | Peptide | Amino Acid Sequence | contiguous; non-contiguous |
| 4 | mPEP35 | LKQKIKHVVKLKVVVKLRSQL | (i) K2-K10, (ii) K10-R18; (iii) K4-K12, (iv) |
| | | VKRKQN | K16-R24 |
| 5 | 17x-3 | KTKATVLIKNKQKSKNALKQKI | (i) K3-K11, (ii) K11-K19, (iii) K19-K27; (iv) |
| | | VLLSK | K1-K9, (v) K13-K21 |
| 6 | 6f-2 | TKSKIKIVVKSKAKKLRLALVK | (i) K6-K14, (ii) K14-K22; (iii) K2-K10, (iv) |
| | | RIKI | K4-K12, (v) K15-R23, |
| | | | (vi) R17-K25 |
| 7 | 8h-2 | TKTKIKIIVKLKSKAKLRIKLVK | (i) K2-K10, (ii) K10-R18, (iii) R18-K26 & (iv) |
| | | RHKS | K4-K12, (v) K12-K20; (vi) K6-K14, (vii) |
| | | | K16-R24 |
| 8 | 2^{nd} de | ASNLTKAAKSLKVRVIKKTKQ | (i) K6-R14, (ii) R14-K22; (iii) K9-K17, (iv) |
| | novo | KQVLKVL | K12-K20, (v) K18-K26 |
| 9 | 10K | TKAKIKHSVKLKAKLRIKLVKRI | (i) K2-K10, (ii) K10-K18 & (iii) K6-K14, (iv) |
| | | | K14-R22; (v) K4-K12 |
| 10 | 4 | LKTKIKIIVKTKSSAKLRSKLVN | (i) K2-K10, (ii) K10-R18, (iii) R18-K26 & (iv) |
| | | SHKI | K4-K12, (v) K12-K20 |
| 11 | 6b | LKLKSSHSIKLKVKSKQRSAL | (i) K2-K10, (ii) K10-R18, (iii) R18-K26; (iv) |
| | | VSRQKA | K4-K12 & (v) K16-R24 |
| 12 | 7c | KTKATVKIKNKQKSVNALKQK | (i) K3-K11, (ii) K11-K19, (iii) K19-K27; (iv) |
| | | IVLLSK | K1-K9, (v) K13-K21 |
| 13 | BHP3 | TQLRNKYTFLARARNALAVRT | (i) R4-R12, (ii) R12-R20 and (iii) K6-14R, |
| | | KQNIKS | (iv) R14-K22 |
| 14 | BHP4 | TNLRNKYTFLARARANLAVRN | (i) R4-R12, (ii) R12-R20 and (iii) K6-14R, |
| | | KQNIKS | (iv) R14-K22 |

The ″N-terminal″ of the peptides may further comprise a amine group (NH2) and the ″C-terminal″ of the peptides may further comprise a carboxyl group (COOH).

In some embodiments, GFs conjugated to CBP allows for a higher concentration of GFs on the surface of collagen IV and increases their binding affinity. In some embodiments, VEGF conjugated to CBP allows for a higher concentration of VEGF on the surface of collagen IV and increases its binding affinity. In other embodiments, GFs conjugated to CBP allows for a higher concentration of GFs on the surface of collagen I, collagen III, collagen VI, or a combination thereof and increases their binding affinity. In other embodiments, GFs conjugated to hyaluronic acid (HA) binding peptide allows for a higher concentration of GFs on the surface of HA and increases their binding affinity.

The present invention may feature a composition comprising a growth factor (GF) conjugated to one or more avidin proteins or streptavidin proteins (to create a GF-avidin (or streptavidin composition)). In some embodiments, the composition comprises a GF conjugated to two or more avidin proteins or streptavidin proteins. In other embodiments, the composition comprises a GF conjugated to three or more avidin proteins or streptavidin proteins. In further embodiments, the composition comprises a GF conjugated to four or more avidin proteins or streptavidin proteins. The compositions described herein may comprise a GF conjugated to one, or two, or three, or four avidin proteins or streptavidin proteins.

According to some embodiments, the present invention may feature a composition comprising a vascular endothelial growth factor (VEGF) protein conjugated to one or more avidin proteins or streptavidin proteins (to create a VEGF-avidin (or streptavidin) composition). In other embodiments, the present invention may feature a composition comprising a VEGF protein conjugated to two or more, or three or more, or four or more avidin proteins or streptavidin proteins. In further embodiments, the present invention may feature a composition comprising a VEGF protein conjugated to one, two, three, or four avidin proteins or streptavidin proteins. In some embodiments, the present invention features a composition comprising an ANG1 or ANG2 protein conjugated to one or more avidin proteins or streptavidin proteins. In other embodiments, the present invention features a composition comprising an ANG1 or ANG2 protein conjugated to one, or two, or three, or four avidin proteins or streptavidin proteins.

The present invention may also feature a composition comprising a peptide (e.g., a peptide that mimics bioactivity (e.g., QK peptide)) conjugated to one or more avidin proteins or streptavidin proteins (to create a QK-avidin (or streptavidin) composition). In some embodiments, the composition comprises a peptide (e.g., a peptide that mimics bioactivity (e.g., QK peptide)) conjugated to two or more avidin proteins or streptavidin proteins. In other embodiments, the composition comprises a peptide (e.g., a peptide that mimics bioactivity (e.g., QK peptide)) conjugated to three or more avidin proteins or streptavidin proteins. In further embodiments, the composition comprises a peptide (e.g., a peptide that mimics bioactivity (e.g., QK peptide)) conjugated to four or more avidin proteins or streptavidin proteins. The composition may comprise a peptide (e.g., a peptide that mimics bioactivity (e.g., QK peptide)) conjugated to one, or two, or three, or four avidin proteins or streptavidin proteins.

In some embodiments, the composition further comprises a targeting moiety (e.g., CBP) conjugated to biotin (to create a biotin-targeting moiety composition). The composition may further comprise a multi-antigen peptide (MAP) comprising four lysines conjugated to four targeting moieties (to create a targeting moiety-MAP4 composition). In some embodiments, the targeting moiety is conjugated to both a biotin and a MAP (to create a biotin-targeting moiety-MAP4 composition).

In some embodiments, the composition further comprises a collagen binding protein (CPB) conjugated to biotin (to create a CBP-biotin composition). In other embodiments, the composition further comprises multi-antigen peptide (MAP) comprising four lysines conjugated to four collagen binding proteins (CPB) (to create a CBP-MAP4 composition). In some embodiments, the CBP is conjugated to both biotin and MAP (to create a biotin-CBP-MAP4 composition).

A MAP is a multiple antigen peptide that can be synthesized economically with lysine linkers during solid phase synthesis. This can be done because lysine has a NH2 on is side residue and on its N-terminus allowing for branching to happen during the peptide chemistry. The sequence may be specified, within a reasonable size, to be branched in such a manner to make tetramers (3 lysines) and octomers (5 lysines). Without wishing to limit the present invention to any theories or mechanisms it is believed that using MAPs allows for viable multivalent and/or branched extracellular matrix (ECM) binders with higher affinities. Additionally, MAPs may also be used with biotin to facilitate self-assembled structures or layers with avidin/streptavidin. Conjugation of parts to the biotin-MAPs and avidin can create things like ECM binding growth factor coatings.

In some embodiments, the MAP peptides may be combined (i.e., conjugated) to two or three different biotin-targeting moiety compositions (e.g., a biotin-collagen bind protein (CBP) composition and/or a biotin-hyaluronic acid binding peptide (HBP) composition) (i.e., a singular MAP peptide conjugated to both a biotin-CBP composition and a biotin-HBP composition). The conjugation of a MAP peptide to multiple biotin-targeting moiety compositions may be achieved by using alloc protecting groups during solid phase synthesis. Without wishing to limit the present invention to any theories or mechanisms to it believed that the use of a mixture of MAPs such as a biotin-CBP-MAP4/biotin-HBP-MAP4 composition would create two possible anchoring sites onto collagen IV and hyaluronic acid simultaneously during the self-assembly of the growth-factor coating.

The GF-avidin (or streptavidin) composition may further be combined with the targeting moiety-biotin composition and/or the biotin-targeting moiety-MAP4 composition. In some embodiments, the VEGF-avidin (or streptavidin) composition can be combined with CBP-biotin composition or Biotin-CBP-MAP4 composition. In some embodiments, the ANG1-avidin (or streptavidin) composition can be combined with CBP-biotin composition or Biotin-CBP-MAP4 composition. In some embodiments, the ANG2-avidin (or streptavidin) composition can be combined with CBP-biotin composition or Biotin-CBP-MAP4 composition. Additionally, the QK-avidin (or streptavidin) composition may be combined with the targeting moiety-biotin composition and/or the biotin-targeting moiety-MAP4 composition.

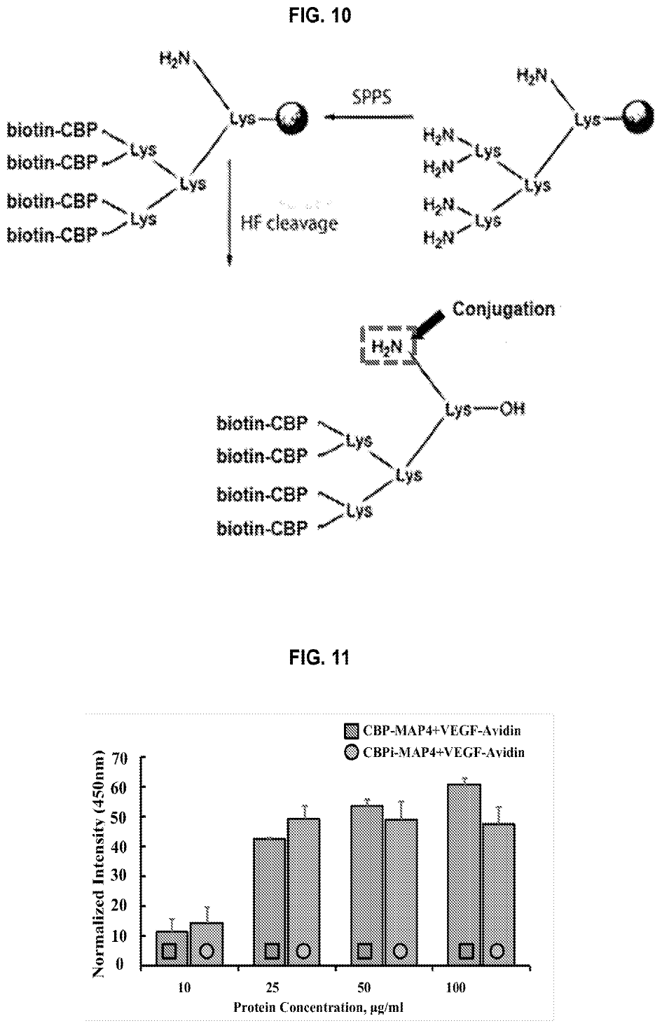

These combinations use the Biotin/Avidin (or streptavidin) interaction, which is one of the most potent intermolecular binding interactions in nature. This dual-assembly complex forms a VEGF layer specifically at collagen IV sites. The MAP4 (Biotin-CBP), is a tetramer (FIG. 10) that binds to collagen IV (at 4 sites, corresponding to the 4 CBP molecules attached) and forms a complex with avidin (also at 4 sites, corresponding to the 4 biotin molecules attached to the CBP) on the VEGF-avidin composition allowing for its accumulation on the collagen IV.

With the CBP-biotin composition, other growth factors (e.g., VEGF, ANG1, or ANG2) and binders can be substituted to make comparative studies simple or more cost effective. Furthermore, the Biotin-CBP-MAP4 composition (see structure in FIG. 10) allows for a self-assembling structure where Biotin-CBP-MAP4 is a tetramer with 4 CBP sequences that targets the collagen IV and 4 biotin that attracts VEGF-Avidin, and the VEGF-Avidin has four active sites that attracts Biotin (see below). This is a crosslinking interaction that builds a 3D VEGF structure onto the collagen IV, which goes beyond the 2D limit of the CBP binding alone.

In some embodiments, the tetramer biotin molecules (either as the CPB-biotin composition or the MAP4-Biotin-CBP composition) in combination with the tetramer avidin molecules (i.e., the VEGF-Avidin composition) allows for a very robust binding. Without wishing to limit the present invention to any theories or mechanisms it is believed that non-bound ECM can self-assemble with the VEGF-Avidin composition to build a potent angiogenic response.

In some embodiments, biotin can be attached to any extracellular matrix (ECM) binder. In some embodiments, biotin is attached to a collagen binding protein (CPB). In some embodiments, avidin is attached to a growth factor. In some embodiments, avidin is attached to a vascular endothelial growth factor (VEGF) protein. In other embodiments, avidin is attached to a bioactive ligand. As used herein, a “bioactive ligand” may refer to growth factors (e.g., VEGF, ANG1, and/or ANG2), biomolecules (e.g., heparin), peptides that mimic bioactivity (e.g., QK peptides), and/or drugs.

In certain embodiments, the GFs (e.g., VEGF, ANG1, and/or ANG2) may be conjugated to one or more biotin molecules, or two or more biotin molecules, or three or more biotin molecules, or four or more biotin molecules. In some embodiments, the targeting moieties (e.g., CBP and/or HBP) may be conjugated to one or more avidin (or streptavidin) proteins, or two or more avidin (or streptavidin) proteins, or three or more avidin (or streptavidin) proteins, or four or more avidin (or streptavidin) proteins.

The present invention may additionally feature a composition comprising heparin conjugated to one or more targeting agents. In some embodiments, the composition comprises heparin conjugated to two or more targeting agents, or three or more targeting agents, or four or more targeting agents. In some embodiments, the composition comprises a portion of heparin conjugated to one or more targeting agents, or two or more targeting agents, or three or more targeting agents, or four or more targeting agents. In some embodiments, the composition comprises heparin or a portion thereof conjugated to one targeting moiety, or two targeting moieties, or three targeting moieties, or four targeting moieties.

Furthermore, compositions described herein may further comprise heparin or a portion thereof conjugated to a biotin-targeting moiety-MAP4 composition (to create a biotin-targeting moiety-MAP4-heparin composition). In some embodiments, the composition comprises heparin or a portion thereof conjugated to a biotin-CBP-MAP composition (to create a biotin-CBP-MAP-heparin composition).

In some embodiments, heparin or a portion thereof may be conjugated to one or more avidin proteins or streptavidin proteins (to create a heparin-avidin (or streptavidin) composition). In other embodiments, heparin or a portion thereof may be conjugated to two or more avidin proteins or streptavidin proteins, or three or more avidin proteins or streptavidin proteins, or four or more avidin proteins or streptavidin proteins. In further embodiments, heparin or a portion thereof may be conjugated to one, or two, or three, or four avidin proteins or streptavidin proteins. The heparin-avidin (or streptavidin) composition may further be combined with a biotin-targeting moiety-MAP4 (e.g., a biotin-CBP-MAP4) composition described herein.

Heparin is a glycosaminoglycan (i.e., a sugar) and is a potent modulator for growth factors and clotting. In some embodiments, the use of heparin in compositions described herein may increase VEGF secretion from endothelial cells. In other embodiments, the use of heparin in compositions described herein may help form nanofibers in a self-supporting gel that promotes angiogenesis.

According to other embodiments, the present invention also features methods for forming a vascular system in an artificial kidney. In some embodiments, the method may be used to form a vascular system in an artificial organ that requires vasculature including but not limited to an artificial kidney, an artificial liver, an artificial heart, or an artificial lung. In further embodiments, the method may be used to form a vascular system in artificial tissue including but not limited to artificial connective tissue or artificial bone tissue. The method may comprise introducing one or more of the compositions described herein to the artificial organ and/or tissue. In some embodiments, the present invention features a method of forming a vascular system in an artificial kidney, comprising introducing a composition described herein to the artificial kidney.

The present invention may also feature methods of forming vascular within a scaffold structure (e.g., a scaffold for creating an artificial organ). In some embodiments, the method comprises introducing a composition described herein to the scaffold structure. In other embodiments, the method comprises introducing one or more compositions described herein to the scaffold structure. In further embodiments, the method comprises introducing two or more compositions described herein to the scaffold structure. The present invention may also be used to promote the rebuilding of vasculature on a de-celled organs (e.g., a kidney) or artificial organs.

In some embodiments, the methods and compositions described herein are able to recreate vasculature that is the same pattern as collagen IV on a scaffold and/or an artificial organ. For example, a 3D printed pattern with collagen IV could be mixed with the compositions described herein to attract endothelial cells and promote angiogenesis. In other embodiments, the methods and compositions described herein are able to recreate vasculature that is the same pattern as collagen I, collagen III, collagen IV, collagen VI, or a combination thereof on a scaffold and/or an artificial organ. In further embodiments, the methods and compositions described herein are able to recreate vasculature that is the same pattern as hyaluronic acid and/or heparin on a scaffold and/or an artificial organ.

The present invention may also feature methods of localizing a growth factor to a scaffold. The method may comprise conjugating a growth factor to one or more targeting moieties. In some embodiments, the growth factor is a vascular endothelial growth factor (VEGF) protein. In some embodiments, any scaffold with collagen IV present can be used for the methods described herein. In other embodiments, a scaffold has collagen IV mixed into it may be used for the methods described herein.

In some embodiments, the scaffold comprises endogenous collagen IV. In other embodiments, the scaffold comprises exogenous collagen IV. In further embodiments, the scaffold is de-celled and comprises endogenous collagen IV. In some embodiments, the scaffold comprises extracellular matrix (ECM) components.

In other embodiments, the present invention features a method of localizing a vascular endothelial growth factor (VEGF) protein to a scaffold. The method comprises conjugating VEGF to a targeting moiety and/or introducing said composition to the scaffold. In further embodiments, the present invention features localizing a QK peptide to a scaffold. The method comprises conjugating a QK peptide to a targeting moiety and/or introducing said composition to the scaffold. In some embodiments, the method comprises introducing to the scaffold a composition as described herein.

In some embodiments, the methods and compositions described herein can be used to localize and concentration GF (e.g., VEGF, ANG1, or ANG2). In other embodiments, the methods and compositions described herein can be used to localize and concentration VEGF. In some embodiments, the correct localization of VEGF allows for the recruitment of cells for angiogenesis. In other embodiments, concentrated VEGF allows for proper signaling to cue the start of the process of angiogenesis. In other embodiments, the localization and concentration of VEGF can be used in conjunction with de-cell organs (e.g., de-celled kidneys) or artificial organs (e.g., artificial kidneys) to allow for proper vasculature and blood circulation.

The present invention may further feature methods of synthesizing compositions described herein.

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

1.-75. (canceled)

76. A composition comprising a growth factor (GF) protein conjugated to one or more targeting moieties.

77. The composition of claim 76 further comprising two or more targeting moieties.

78. The composition of claim 76 further comprising three or more targeting moieties.

79. The composition of claim 76, wherein the GF is a vascular endothelial growth factor (VEGF) protein.

80. The composition of claim 79, wherein the VEGF protein is an isoform of VEGF, wherein the isoform of VEGF is VEGF 121 or VEGF 165.

81. The composition of claim 76, wherein the GF is an angiopoietin-1 (ANG1) protein or an angiopoietin-2 (ANG2) protein.

82. The composition of claim 76, wherein the targeting moiety is a collagen-binding protein (CBP), wherein the CBP is selected from SEQ ID NO: 1, SEQ ID NO: 2, or SEQ ID NO: 3.

83. The composition of claim 76, wherein the targeting moiety is a hyaluronic acid binding peptide (HBP), wherein the HBP is a mPep35 peptide, a 17x3 peptide, a 17x4 peptide, a BHP3 peptide, or a BHP4 peptide.

84. The composition of claim 83, wherein the HBP is selected from SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 14.

85. The composition of claim 76, wherein conjugation occurs via Click-Chemistry.

86. A composition comprising
a. a growth factor (GF) conjugated to one or more avidin or streptavidin proteins and
b. one or more targeting moieties, each conjugated to a biotin molecule, wherein the GF is conjugated to the one or more targeting moieties via the interaction between the biotin molecules and the one or more avidin or streptavidin proteins.

87. The composition of claim 86 further comprising two or more targeting moieties.

88. The composition of claim 86 further comprising two or more avidin or streptavidin proteins.

89. The composition of claim 86, wherein the GF is a vascular endothelial growth factor (VEGF) protein.

90. The composition of claim 89, wherein the VEGF protein is an isoform of VEGF, wherein the isoform of VEGF is VEGF 121 or VEGF 165.

91. The composition of claim 86 wherein the GF is an angiopoietin-1 (ANG1) protein, or an angiopoietin-2 (ANG2) protein.

92. The composition of claim 86, wherein the targeting moiety is a collagen binding protein (CBP), wherein the CBP is selected from SEQ ID NO: 1, SEQ ID NO: 2, or SEQ ID NO: 3.

93. The composition of claim 86, wherein the targeting moiety is a hyaluronic acid binding peptide (HBP), wherein the HBP is a mPep35 peptide, a 17x3 peptide, a 17x4 peptide, a BHP3 peptide, or a BHP4 peptide.

94. The composition of claim 93, wherein the HBP is selected from SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 14.

95. The composition of claim 86 further comprising a multi-antigen peptide (MAP) conjugated to the one or more targeting moieties, wherein the MAP comprises four lysines conjugated to four targeting moieties.


SEQUENCE LISTING (16 sequences)

SEQ ID NO: 1 - protein, 10 aa, Unknown
  collagen binding protein (CBP)
CQDSETRTFY

SEQ ID NO: 2 - protein, 9 aa, Unknown
  human decorin collagen binding peptide (CBP)
RELHLNNNL

SEQ ID NO: 3 - protein, 10 aa, Unknown
  bovine CBP
LRELHLNNNC

SEQ ID NO: 4 - protein, 27 aa, Unknown
  mPEP35 peptide
LKQKIKHVVKLKVVVKLRSQLVKRKQN

SEQ ID NO: 5 - protein, 27 aa, Unknown
  17x-3 peptide
KTKATVLIKNKQKSKNALKQKIVLLSK

SEQ ID NO: 6 - protein, 26 aa, Unknown
  6f-2 peptide
TKSKIKIVVKSKAKKLRLALVKRIKI

SEQ ID NO: 7 - protein, 27 aa, Unknown
  8h-2 peptide
TKTKIKIIVKLKSKAKLRIKLVKRHKS

SEQ ID NO: 8 - protein, 28 aa, Unknown
  2nd de novo peptide
ASNLTKAAKSLKVRVIKKTKQKQVLKVL

SEQ ID NO: 9 - protein, 22 aa, Unknown
  10K peptide
TKAKIKHSVKLKAKLRIKLVKR

SEQ ID NO: 10 - protein, 27 aa, Unknown
  4 peptide
LKTKIKIIVKTKSSAKLRSKLVNSHKI

SEQ ID NO: 11 - protein, 27 aa, Unknown
  6b peptide
LKLKSSHSIKLKVKSKQRSALVSRQKA

SEQ ID NO: 12 - protein, 27 aa, Unknown
  7c peptide
KTKATVKIKNKQKSVNALKQKIVLLSK

SEQ ID NO: 13 - protein, 27 aa, Unknown
  BHP3 peptide
TQLRNKYTFLARARNALAVRTKQNIKS

SEQ ID NO: 14 - protein, 27 aa, Unknown
  BHP4 peptide
TNLRNKYTFLARARANLAVRNKQNIKS

SEQ ID NO: 15 - protein, 15 aa, Unknown
  QK peptide
KLTWQELYQLKYKGI

SEQ ID NO: 16 - protein, 22 aa, Unknown
  CBP-QK composition
CQDSETRTKLTWQELYQLKYKG